(19) United States
(12) Patent Application Publication
(10) Pub. No.: US 2025/0361209 A1
(43) Pub. Date: Nov. 27, 2025

(54) CRYSTAL FORMS OF N-(3-FLUOROPHENYL)-6-(6,7-DIMETHOXYQUINOLIN-4-OXY)-3,4-DIHYDROQUINOLINE-1(2H)-CARBOXAMIDE METHANESULFONATE AND PREPARATION METHOD THEREOF

(71) Applicant: Shanghai Yi Feng Biotechnology Co., Ltd., Shanghai (CN)

(72) Inventors: Min GUO, Shanghai (CN); Yidong XING, Shanghai (CN)

(73) Assignee: Shanghai Yi Feng Biotechnology Co., Ltd., Shanghai (CN)

(21) Appl. No.: 19/296,289

(22) Filed: Aug. 11, 2025

Related U.S. Application Data
(63) Continuation of application No. PCT/CN2024/139447, filed on Dec. 16, 2024.

(30) Foreign Application Priority Data
Oct. 19, 2023 (CN) 2023113616415

(51) Int. Cl. C07D 215/22; C07C 309/04

(52) U.S. Cl. CPC C07D 215/22; C07C 309/04; C07B 2200/13

(57) ABSTRACT

The present disclosure belongs to the technical field of medicinal chemistry, and in particular relates to crystalline forms A, B, and C of a compound N-(3-fluorophenyl)-6-(6,7-dimethoxyquinolin-4-oxy)-3,4-dihydroquinoline-1(2H)-carboxamide methanesulfonate and preparation methods thereof. The three crystalline forms A, B, and C provided by the present disclosure can be prepared under various different conditions, a crystallization process has a good purification effect and has advantageous characteristics such as stable process and easy operation, the preparation methods for the crystalline forms are simple and has low cost, and different crystal forms of the compound N-(3-fluorophenyl)-6-(6,7-dimethoxyquinolin-4-oxy)-3,4-dihydroquinoline-1(2H)-carboxamide methanesulfonate with high purity, good solubility and good stability can be obtained.

### FIELD OF THE INVENTION

The present disclosure belongs to the field of medicinal chemistry, and in particular relates to crystalline forms A, B, and C of N-(3-fluorophenyl)-6-(6,7-dimethoxyquinolin-4-oxy)-3,4-dihydroquinoline-1(2H)-carboxamide methanesulfonate and preparation methods thereof.

### BACKGROUND OF THE INVENTION

Malignant tumors are one of the major diseases that severely affect human health and threaten human life. The World Health Organization and health departments of governments around the world have listed overcoming cancer as a primary task. At present, the commonly used anticancer drugs in clinical practice are mainly cytotoxic drugs, which have unavoidable disadvantages of poor selectivity, strong toxic and side effects, and easy development of drug resistance due to their inherent properties of cytotoxicity. Therefore, it is an urgent need for anticancer drug research to find new targets with high specificity, low toxicity and good patient tolerance. In recent years, with the rapid development of life science research, many specific targets based on the mechanisms of cancer cell occurrence and development, such as vascular endothelial cell growth factors (VEGFR1, VEGFR2, and VEGFR3) which inhibit tumor angiogenesis, have been identified. Angiogenesis refers to the development of a new vascular system from existing blood vessels. Normal angiogenesis occurs only during certain short-term and specific physiological processes, such as reproduction, wound healing, and the like. Abnormal angiogenesis is one of the pathological manifestations of malignant diseases such as tumors, rheumatoid arthritis and diabetic retinopathy. Since Folkman put forward the hypothesis that angiogenesis is closely related to the occurrence and development of tumors, a large number of clinical practices and experimental studies have confirmed that inhibiting tumor-mediated angiogenesis can effectively inhibit tumor growth and metastasis.

VEGF receptors are important targets for anti-angiogenesis. In recent years, the research of small molecule inhibitors targeting the VEGF receptors has been very active, and a large number of inhibitors with different structures have been reported. However, at present, these inhibitors still have some problems. For example, they are all competitive inhibitors of ATP, and the concentration of ATP in cells, especially in cancer cells, can reach 5 mmol/L or more. Therefore, the inhibitor activity should reach at least a nanomolar level in order to exhibit effective inhibitory effects. In addition, the VEGF receptors belong to the tyrosine kinase superfamily, members of which are widely involved in the transduction of biological signals in vivo. Due to sequence homology, the three-dimensional structure of their ATP binding sites is highly conserved. Therefore, how to improve the selectivity of the inhibitors among these family members is extremely important. Chinese invention patent No. CN103524409A discloses a class of quinoline tyrosine kinase inhibitors, which generally have good tyrosine kinase inhibitory activity in vitro, and in particular, have good inhibitory activity on VEGFR2 and VEGFR3, but the druggability of salt forms and crystalline forms of specific compounds has not been further studied.

Described in the present disclosure is a compound N-(3-fluorophenyl)-6-(6,7-dimethoxyquinolin-4-oxy)-3,4-dihydroquinoline-1(2H)-carboxamide methanesulfonate, which exhibits outstanding VEGFR2 and VEGFR3 inhibitory activity. Further research has shown that the methanesulfonate of this product can improve the physicochemical or biological properties of a drug, and can achieve faster dissolution and release in the body than a free base, which is conducive to the absorption and the exertion of drug efficacy in the human body, and has more clinical advantages.

In view of the importance of solid drug crystalline forms and their stability in clinical treatment, in-depth research on polymorphic forms of the compound N-(3-fluorophenyl)-6-(6,7-dimethoxyquinolin-4-oxy)-3,4-dihydroquinoline-1(2H)-carboxamide methanesulfonate is of great significance for the development of drugs suitable for industrial production and with good biological activity.

### SUMMARY OF THE INVENTION

In view of the problems such as solubility in water, stability and oral bioavailability of the compound, the inventors have found that N-(3-fluorophenyl)-6-(6,7-dimethoxyquinolin-4-oxy)-3,4-dihydroquinoline-1(2H)-carboxamide methanesulfonate can solve the problems after long-term efforts.

The present disclosure further provides a compound N-(3-fluorophenyl)-6-(6,7-dimethoxyquinolin-4-oxy)-3,4-dihydroquinoline-1(2H)-carboxamide methanesulfonate, having the following structural formula:

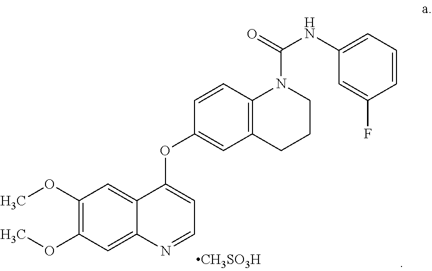

Further, a crystalline form A of the compound N-(3-fluorophenyl)-6-(6,7-dimethoxyquinolin-4-oxy)-3,4-dihydroquinoline-1(2H)-carboxamide methanesulfonate has characteristic peaks in an X-ray powder diffraction pattern at 2θ of 6.5°±0.2°, 10.0°±0.2°, 15.2°±0.2°, 17.2°±0.2°, 19.8°±0.2°, and 24.3°±0.2°. Preferably, the crystalline form A has characteristic peaks at 2θ of 6.5984, 10.0740, 15.2443, 17.2032, 19.8381, and 24.3214. Preferably, the crystalline form A has characteristic peaks at 2θ of 6.5984, 7.6194, 10.0740, 13.3938, 15.2443, 17.2032, 18.8396, 19.8381, and 24.3214. Preferably, the crystalline form A has characteristic peaks at 2θ of 6.5984, 7.6194, 10.0740, 13.1889, 13.3938, 15.2443, 17.2032, 18.8396, 19.8381, 24.3214, 25.8243, and 27.6430.

The present disclosure also provides a method for preparing the crystalline form A of the compound, including:
weighting the compound N-(3-fluorophenyl)-6-(6,7-dimethoxyquinolin-4-oxy)-3,4-dihydroquinoline-1(2H)-carboxamide methanesulfonate into a vial, dissolving the compound by adding a solvent to be clear, and slowly volatilizing the solvent at room temperature.

Further, in the above step, the solvent is Acetone/H₂O, EtOH/H₂O, methanol/water, THF/H₂O or ACN/H₂O in a volume ratio of 3-4:1, and a volume (ml) of the solvent used is 0.1-0.5 times a weight (mg) of the compound.

Further, the present disclosure provides a crystalline form B of the compound N-(3-fluorophenyl)-6-(6,7-dimethoxyquinolin-4-oxy)-3,4-dihydroquinoline-1(2H)-carboxamide methanesulfonate, having characteristic peaks in an X-ray powder diffraction pattern at 2θ of 5.3°±0.2°, 9.6°±0.2°, 14.5°±0.2°, 21.5°±0.2°, 24.1°±0.2°, and 27.0°±0.2°. Preferably, the crystalline form B has characteristic peaks at 2θ of 5.3766, 9.6473, 14.5320, 21.5785, 24.1021, and 27.0263. Preferably, the crystalline form B has characteristic peaks at 2θ of 5.3766, 8.4489, 9.6473, 11.3723, 14.5320, 17.0842, 21.5785, 24.1021, and 27.0263. Preferably, the crystalline form B has characteristic peaks at 2θ of 5.3766, 8.4489, 9.6473, 10.7522, 11.3723, 14.5320, 15.6624, 17.0842, 21.5785, 24.1021, 27.0263, and 29.8914.

Further, a method for preparing the crystalline form B includes:
weighting the compound N-(3-fluorophenyl)-6-(6,7-dimethoxyquinolin-4-oxy)-3,4-dihydroquinoline-1(2H)-carboxamide methanesulfonate into a vial, dissolving the compound by adding a solvent, performing filtering, and allowing a filtrate and an anti-solvent to each independently coexist in a closed space and stand at room temperature.

Further, in the above step, the solvent is selected from DMF, CHCl₃, and methanol, the anti-solvent is selected from Acetone, THF, and MEK, and a volume (ml) of the solvent used is 0.04-0.1 times a weight (mg) of the compound, and a volume (ml) of the anti-solvent used is 1-5 times the volume (ml) of the solvent.

Further, the present disclosure provides a crystalline form C of the compound N-(3-fluorophenyl)-6-(6,7-dimethoxyquinolin-4-oxy)-3,4-dihydroquinoline-1(2H)-carboxamide methanesulfonate, having characteristic peaks in an X-ray powder diffraction pattern at 2θ of 4.7°±0.2°, 10.4°±0.2°, 15.7°±0.2°, 19.8°±0.2°, 22.8°±0.2°, and 25.8°±0.2°. Preferably, the crystalline form C has characteristic peaks at 2θ of 4.7539, 10.4270, 15.7866, 19.8422, 22.8579, and 25.8347. Preferably, the crystalline form C has characteristic peaks at 2θ of 4.7539, 9.4706, 10.4270, 12.6174, 15.7866, 17.6406, 19.8422, 22.8579, and 25.8347. Preferably, the crystalline form C has characteristic peaks at 2θ of 4.7539, 9.4706, 10.4270, 11.0858, 12.6174, 14.0934, 15.7866, 17.6406, 19.8422, 22.8579, 25.8347, and 27.6134.

Further, a method for preparing the crystalline form C includes:
weighting the compound N-(3-fluorophenyl)-6-(6,7-dimethoxyquinolin-4-oxy)-3,4-dihydroquinoline-1(2H)-carboxamide methanesulfonate into a vial, adding a solvent, and performing pulping.

Further, in the step, the solvent is selected from IPA, MEK, IPAc, 1-PrOH, ethanol, methanol, Acetone, 2-MeTHF, EtOAc, MTBE, ACN, 1,4-Dioxane, THF, DCM, MIBK, Anisole, n-BuOH, or a mixed solvent NMP/Anisole, DMAc/n-Hexane, DMSO/MEK, Acetone/H₂O, Acetone/EtOH, DMF/Toluene, CHCl₃/n-Heptane, EtOH/H₂O, NMP/EtOAc, DMSO/Toluene, DMF/MIBK, CHCl₃/THF, or MeOH/CPME, a volume (ml) of the solvent or the mixed solvent used is 0.025 times a weight (g) of the compound, a volume ratio (ml/ml) of Acetone to H₂O in the mixed solvent is 1.5:1-75:1, and a volume ratio in other mixed solvents is 1:4-4:1.

Further, a method for preparing the crystalline form C includes:
weighting the compound N-(3-fluorophenyl)-6-(6,7-dimethoxyquinolin-4-oxy)-3,4-dihydroquinoline-1(2H)-carboxamide methanesulfonate into a vial, dissolving the compound by adding a solvent, performing filtering, and allowing a filtrate and an anti-solvent to each independently coexist in a closed space and stand at room temperature.

Further, in the step, the solvent is selected from DMF, CHCl₃, and methanol, the anti-solvent is selected from Ethyl formate, IPAc, IPA, MTBE, and ACN, a volume (ml) of the solvent used is 0.04-0.1 times a weight (mg) of the compound, and a volume (ml) of the anti-solvent used is 1-5 times the volume (ml) of the solvent.

Further, a method for preparing the crystalline form C includes:
weighting the compound N-(3-fluorophenyl)-6-(6,7-dimethoxyquinolin-4-oxy)-3,4-dihydroquinoline-1(2H)-carboxamide methanesulfonate into a vial, dissolving the compound by adding a solvent, adding an anti-solvent, and performing stirring until a solid is separated out.

Further, in the step, the solvent is selected from DMSO, NMP, methanol, and DCM, the anti-solvent is selected from Toluene, IPA, MEK, MTBE, Acetone, n-BuOH, and Anisole, a volume (ml) of the solvent used is 0.05-0.15 times a weight (mg) of the compound, and a volume (ml) of the anti-solvent used is 2-10 times the volume (ml) of the solvent.

A total of three crystalline forms of methanesulfonate, which are crystalline forms A/B/C of methanesulfonate, respectively, are found during screening and repeated preparation, and representative samples of the obtained crystalline forms of methanesulfonate are characterized and identified by using methods such as XRPD, TGA, DSC, and high-performance liquid chromatography (HPLC). The results show that the crystalline forms B and C of methanesulfonate are anhydrous crystalline forms and the crystalline form A of methanesulfonate is a hydrate.

A conversion relationship between the anhydrous crystalline forms B/C of methanesulfonate and the crystalline form A of methanesulfonate in the form of the hydrate is studied by a suspension competition test. The results show that in the suspension competition test of the anhydrous crystalline forms B/C of methanesulfonate, the anhydrate crystalline form C is obtained in both Acetone (5° C., RT and 50° C.) and IPAc (RT and 50° C.) systems; and in the suspension competition test of the anhydrous crystalline forms B/C of methanesulfonate and the crystalline form A of methanesulfonate in the form of the hydrate, the anhydrous crystalline form C is obtained in an Acetone/water system with a water activity (a_{w}) of 0-0.6 at room temperature. The crystalline form A of methanesulfonate is converted into an amorphous state at a high temperature of 150° C., and is continued to be heated to 190° C. and cooled to 30° C. to be converted into the crystalline form C.

It should be understood that slightly different melting point readings may be given with different types of equipment or with different test conditions. The correct values for melting points of different crystalline forms will be affected by the purity of the compound, the sample weight, a heating rate, a particle size, and testing equipment verification and maintenance. Numerical values provided cannot be taken as absolute values.

It should be understood that slightly different XPRD patterns and peaks may be given with different types of equipment or with different test conditions. The patterns, peaks and the relative intensities of diffraction peaks of different crystalline forms will be affected by the purity of the compound, the pretreatment of the sample, a scanning speed, a particle size and testing equipment verification and maintenance. Numerical values provided cannot be taken as absolute values.

The “X-ray powder diffraction pattern or XPRD” in the present disclosure is obtained by Cu-Kα ray diffraction.

“Differential Scanning calorimetry or DSC” in the present disclosure refers to measuring a temperature difference and a heat flow difference between a sample and a reference substance during the temperature rise or constant temperature process of the sample, so as to characterize all physical changes and chemical changes related to the thermal effect, and obtain phase change information of the sample.

A diffraction angle 2θ in the present disclosure is a Bragg angle in degrees, and an error range of 2θ is ±0.2.

The beneficial effects of the present disclosure are that the crystalline forms A, B and C of the compound provided by the present disclosure has more advantages in terms of stability, solubility, and dissolution of a formulation, is more suitable for drug development, meets the requirements of oral bioavailability and drug efficacy, and can meet pharmaceutical requirements for production, transportation and storage, and a production process is stable, repeatable and controllable, and can be adapted to industrial production.

### BRIEF DESCRIPTION OF DRAWINGS

FIG. 1 shows an XPRD pattern of a crystalline form A of a compound;

FIG. 2 shows TGA and DSC patterns of the crystalline form A of the compound;

FIG. 3 shows an XPRD pattern of a crystalline form B of the compound;

FIG. 4 shows TGA and DSC patterns of the crystalline form B of the compound;

FIG. 5 shows an XPRD pattern of a crystalline form C of the compound; and

FIG. 6 shows TGA and DSC patterns of the crystalline form C of the compound.

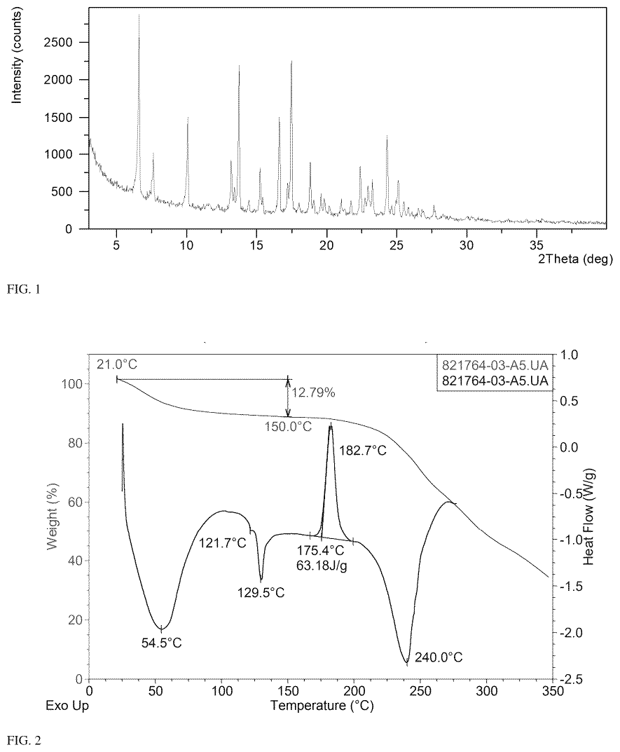

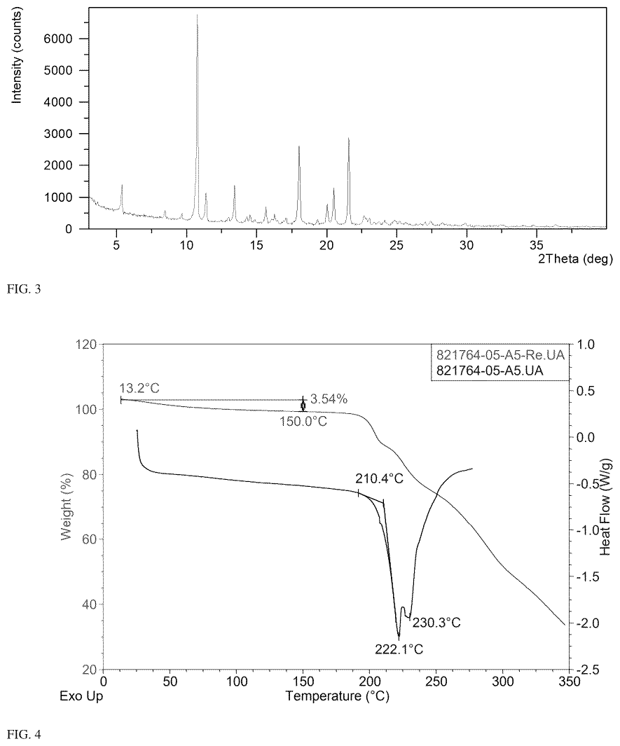

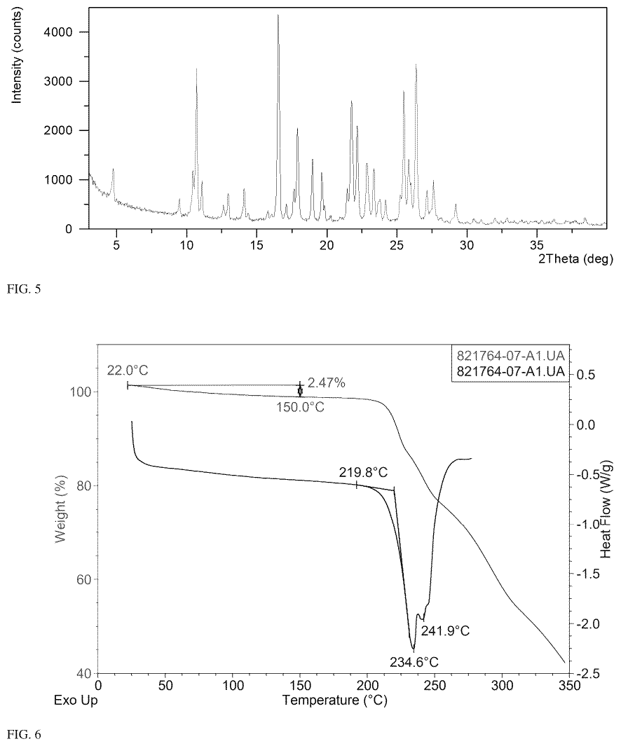

### DETAILED DESCRIPTION OF THE INVENTION

The present disclosure is further described in detail below with reference to the examples, but is not limited thereto.

### Test Conditions of Instruments Used in the Experiments

XRPD is X-ray powder diffraction detection: determination was carried out by using PANalytical Empyrean and an X'Pert3 X-ray diffractometer according to General chapter 0451, Volume IV, Chinese Pharmacopoeia (2020 Edition) under test conditions: Target: Cu; 45 kv, 40 mA.

TGA thermogravimetric analysis and DSC differential scanning calorimetry: determination was carried out by using a TA Discovery 5500 thermogravimetric analyzer and a TA Discovery 2500 differential scanning calorimeter according to General chapter 0661, Volume IV, Chinese Pharmacopoeia (2020 Edition) under test conditions: DSC: 30° C. 10° C./min 300° C.; TGA: 30° C. 10° C./min 350° C.

### Example 1 Screening of N-(3-fluorophenyl)-6-(6,7-dimethoxyquinolin-4-oxy)-3,4-dihydroquinoline-1(2H)-carboxamide Salt Types

14 different acids were selected, each forming a salt in the following solvents,

| | | | | Acetone/H₂O |
|---|---|---|---|---|
| Acid | EtOH | EtOAc | THF | (19:1, v/v) |
| Hydrochloric | Low crystallinity | Low crystallinity | Low crystallinity | Being in a free |
| acid | | | | state and no salt |
| | | | | formation |
| Sulfuric acid | Low crystallinity | Low crystallinity | Low crystallinity | Low crystallinity |
| L-aspartic acid | Being in a free | Being in a free | Being in a free | Being in a free |
| | state and no salt | state and no salt | state and no salt | state and no salt |
| | formation | formation | formation | formation |
| Methanesulfonic | Good crystallinity | Good crystallinity | Good crystallinity | Good crystallinity |
| acid | | | | |
| Phosphoric acid | Low crystallinity | Low crystallinity | Low crystallinity | Being in a free |
| | | | | state and no salt |
| | | | | formation |
| Fumaric acid | Being in a free | Being in a free | Being in a free | Being in a free |
| | state and no salt | state and no salt | state and no salt | state and no salt |
| | formation | formation | formation | formation |
| L-tartaric acid | Low crystallinity | Amorphous | Amorphous | Being in a free |
| | | | | state and no salt |
| | | | | formation |
| Citric acid | Being in a free | Being in a free | Amorphous | Being in a free |
| | state and no salt | state and no salt | | state and no salt |
| | formation | formation | | formation |
| Lactic acid | Being in a free | Being in a free | Being in a free | Being in a free |
| | state and no salt | state and no salt | state and no salt | state and no salt |
| | formation | formation | formation | formation |
| Succinic acid | Being in a free | Being in a free | Being in a free | Being in a free |
| | state and no salt | state and no salt | state and no salt | state and no salt |
| | formation | formation | formation | formation |
| Acetic acid | Being in a free | Being in a free | Being in a free | Being in a free |
| | state and no salt | state and no salt | state and no salt | state and no salt |
| | formation | formation | formation | formation |
| Gentisic acid | Being in a free | Being in a free | Being in a free | Being in a free |
| | state and no salt | state and no salt | state and no salt | state and no salt |
| | formation | formation | formation | formation |
| Benzoic acid | Being in a free | Being in a free | Being in a free | Being in a free |
| | state and no salt | state and no salt | state and no salt | state and no salt |
| | formation | formation | formation | formation |
| Malic acid | Being in a free | Amorphous | Being in a free | Being in a free |
| | state and no salt | | state and no salt | state and no salt |
| | formation | | formation | formation |

The inventors have surprisingly found that methanesulfonate has outstanding performance in terms of salt formation and crystallinity.

### Example 2 Preparation of crystalline forms of N-(3-fluorophenyl)-6-(6,7-dimethoxyquinolin-4-oxy)-3,4-dihydroquinoline-1(2H)-carboxamide methanesulfonate

### 1. Preparation of a Crystalline Form A

Approximately 20 mg of each starting sample of K-13 methanesulfonate (N-(3-fluorophenyl)-6-(6,7-dimethoxyquinolin-4-oxy)-3,4-dihydroquinoline-1(2H)-carboxamide methanesulfonate) was weighed separately into 3 mL vials, 2.0-3.0 mL of a solvent was added separately to dissolve solids, after filtration through a filter membrane, the resulting clear filtrate was sealed by a sealing film, a small hole was formed in the sealing film by piercing, and the solvent was allowed to slowly volatilize at room temperature. The solids obtained after volatilization were collected and tested by XRPD to obtain the crystalline form A. The experiments were as follows:

| Solvent (v:v) | Solvent amount (mL) | Result |
|---|---|---|
| EtOH/H₂O (4:1) | 2.5 | Crystalline form A of |
| | | methanesulfonate |
| THF/H₂O (4:1) | 2.5 | Crystalline form A of |
| | | methanesulfonate |
| ACN/H₂O (4:1) | 2.5 | Crystalline form A of |
| | | methanesulfonate |
| Acetone/H₂O (4:1) | 2.5 | Crystalline form A of |
| | | methanesulfonate |

The results of XRPD showed that no change in crystalline form was found when the crystalline form A of methanesulfonate was air-dried at room temperature. The results of TGA showed that when the sample was heated from room temperature to 150° C., a weight loss of the sample was 12.8%. The results of DSC showed that four endothermic peaks were observed at 54.5° C., 121.7° C., 129.5° C. and 240.0° C. (peak temperatures) and one exothermic peak was observed at 182.7° C. (a peak temperature) for this sample.

### XRPD Peak-Searching Data for the Crystalline Form A of Methanesulfonate

| | | FWHM | | Rel. |
|---|---|---|---|---|
| Pos. [°2θ] | Height [cts] | Left [°2θ] | d-spacing [Å] | Int. [%] |
| 6.5984 | 2414.96 | 0.0768 | 13.40 | 100.00 |
| 7.6194 | 633.24 | 0.0768 | 11.60 | 26.22 |
| 10.0740 | 1171.51 | 0.0768 | 8.78 | 48.51 |
| 13.1889 | 669.29 | 0.0768 | 6.71 | 27.71 |
| 13.3938 | 327.74 | 0.0768 | 6.61 | 13.57 |
| 13.7303 | 1927.96 | 0.1023 | 6.45 | 79.83 |
| 14.4234 | 142.67 | 0.1023 | 6.14 | 5.91 |
| 15.2443 | 551.07 | 0.0768 | 5.81 | 22.82 |
| 15.4252 | 201.95 | 0.0768 | 5.74 | 8.36 |
| 16.6182 | 1225.78 | 0.1023 | 5.33 | 50.76 |
| 17.2032 | 392.37 | 0.0768 | 5.15 | 16.25 |
| 17.4747 | 1962.92 | 0.1023 | 5.08 | 81.28 |
| 18.0104 | 127.60 | 0.1535 | 4.93 | 5.28 |
| 18.8396 | 706.43 | 0.0768 | 4.71 | 29.25 |
| 19.0807 | 189.59 | 0.0768 | 4.65 | 7.85 |
| 19.5889 | 269.60 | 0.0768 | 4.53 | 11.16 |
| 19.8381 | 194.01 | 0.1023 | 4.48 | 8.03 |
| 20.2096 | 120.64 | 0.1023 | 4.39 | 5.00 |
| 21.0300 | 177.52 | 0.0768 | 4.22 | 7.35 |
| 21.7355 | 194.08 | 0.1023 | 4.09 | 8.04 |
| 22.4057 | 655.89 | 0.1023 | 3.97 | 27.16 |
| 22.7337 | 225.80 | 0.0768 | 3.91 | 9.35 |
| 22.9484 | 386.43 | 0.0768 | 3.88 | 16.00 |
| 23.2651 | 469.05 | 0.1023 | 3.82 | 19.42 |
| 24.3214 | 988.75 | 0.1023 | 3.66 | 40.94 |
| 25.1059 | 492.01 | 0.1023 | 3.55 | 20.37 |
| 25.5183 | 199.72 | 0.1279 | 3.49 | 8.27 |
| 25.8243 | 131.21 | 0.1023 | 3.45 | 5.43 |
| 26.5561 | 116.48 | 0.1535 | 3.36 | 4.82 |
| 27.6430 | 171.98 | 0.1023 | 3.23 | 7.12 |
| 30.1669 | 34.12 | 0.8187 | 2.96 | 1.41 |

### 2. Preparation of a Crystalline Form B

### Gas-Liquid Diffusion

Approximately 20 mg of each K-13 methanesulfonate was weighed into a 3 mL vial, 1.0-2.0 mL of a solvent was used to dissolve solids, and a clear solution was obtained after filtration by a filter membrane. Approximately 4 mL of an anti-solvent was added into another a 20 mL vial. After the 3 mL vial containing a filtrate was placed open in the 20 mL vial, the 20 mL vial was sealed and allowed to stand at room temperature. When precipitation of a solid was observed, the solid was collected and tested by XRPD to obtain the crystalline form B. The experiments were as follows:

| Solvent (mL) | Anti-solvent (mL) | Result |
|---|---|---|
| DMF (1.0) | Acetone (4.0) | Crystalline form B of methanesulfonate |
| CHCl₃ (2.0) | THF (4.0) | Crystalline form B of methanesulfonate |
| MeOH (2.0) | MEK (4.0) | Crystalline form B of methanesulfonate |

The results of XRPD showed that no change in crystalline form was found before and after air drying the crystalline form B of methanesulfonate at room temperature. The results of TGA showed that when the sample was heated from room temperature to 150° C., a weight loss of the sample was 3.5%. The results of DSC showed that overlapping endothermic peaks were observed at 222.1° C. and 230.3° C. (peak temperatures) for this sample.

### XRPD Peak-Searching Data for the Crystalline Form B of Methanesulfonate

| | | FWHM | | Rel. |
|---|---|---|---|---|
| Pos. [°2θ] | Height [cts] | Left [°2θ] | d-spacing [Å] | Int. [%] |
| 5.3766 | 866.77 | 0.1023 | 16.44 | 13.16 |
| 8.4489 | 259.05 | 0.0768 | 10.47 | 3.93 |
| 9.6473 | 182.65 | 0.0768 | 9.17 | 2.77 |
| 10.7522 | 6585.14 | 0.0768 | 8.23 | 100.00 |
| 11.3723 | 881.77 | 0.0768 | 7.78 | 13.39 |
| 13.4183 | 1173.90 | 0.1023 | 6.60 | 17.83 |
| 14.2755 | 162.69 | 0.1023 | 6.20 | 2.47 |
| 14.5320 | 238.03 | 0.1023 | 6.10 | 3.61 |
| 15.6624 | 503.69 | 0.0768 | 5.66 | 7.65 |
| 16.2772 | 285.68 | 0.0768 | 5.45 | 4.34 |
| 17.0842 | 182.54 | 0.0768 | 5.19 | 2.77 |
| 18.0414 | 2376.64 | 0.1279 | 4.92 | 36.09 |
| 19.3582 | 151.27 | 0.1023 | 4.59 | 2.30 |
| 20.0382 | 615.80 | 0.1279 | 4.43 | 9.35 |
| 20.5095 | 1110.24 | 0.1023 | 4.33 | 16.86 |
| 21.5785 | 2685.34 | 0.1023 | 4.12 | 40.78 |
| 22.6598 | 284.42 | 0.1279 | 3.92 | 4.32 |
| 23.0539 | 204.60 | 0.1023 | 3.86 | 3.11 |
| 24.1021 | 111.29 | 0.2558 | 3.69 | 1.69 |
| 24.8816 | 141.75 | 0.2558 | 3.58 | 2.15 |
| 27.0263 | 112.69 | 0.1023 | 3.30 | 1.71 |
| 27.4137 | 133.31 | 0.1535 | 3.25 | 2.02 |
| 28.2506 | 86.93 | 0.1535 | 3.16 | 1.32 |
| 29.8914 | 88.25 | 0.1535 | 2.99 | 1.34 |
| 32.6423 | 48.88 | 0.3582 | 2.74 | 0.74 |
| 34.7656 | 65.34 | 0.1535 | 2.58 | 0.99 |
| 36.3597 | 76.19 | 0.2047 | 2.47 | 1.16 |

### 3. Preparation of a Crystalline Form C

### 3.1 Gas-Solid Diffusion

A plurality of gas-solid diffusion tests were set up by using different solvents. Approximately 20 mg of each K-13 methanesulfonate was weighed into a 3 mL vial, about 3 mL of a solvent was added to a 20 mL vial, and after the 3 mL vial was placed open in the 20 mL vial, the 20 mL vial was sealed. A solid was collected after standing at room temperature for about 20 days and tested by XRPD to obtain the crystalline form C. The experiments were as follows:

| Solvent | Result |
|---|---|
| DCM | Crystalline form C of methanesulfonate |
| THF | Crystalline form C of methanesulfonate |
| MeOH | Crystalline form C of methanesulfonate |
| CHCl₃ | Crystalline form C of methanesulfonate |

### 3.2 Gas-Liquid Diffusion

Approximately 20 mg of each K-13 methanesulfonate was weighed into a 3 mL vial, 1.0-2.0 mL of a solvent was used to dissolve solids, and a clear solution was obtained after filtration by a filter membrane. Approximately 4 mL of an anti-solvent was added into another a 20 mL vial. After the 3 mL vial containing a filtrate was placed open in the 20 mL vial, the 20 mL vial was sealed and allowed to stand at room temperature. When precipitation of a solid was observed, the solid was collected and tested by XRPD to obtain the crystalline form C. The experiments were as follows:

| | Anti- | |
|---|---|---|
| Solvent (mL) | solvent (mL) | Result |
| DMF (1.0) | IPAc (4.0) | Crystalline form C of methanesulfonate |
| DMF (1.0) | ACN (4.0) | Crystalline form C of methanesulfonate* |
| CHCl₃ (2.0) | Ethyl | Crystalline form C of methanesulfonate |
| | formate (4.0) | |
| CHCl₃ (2.0) | IPA (4.0) | Crystalline form C of methanesulfonate |
| MeOH (2.0) | MTBE (4.0) | Crystalline form C of methanesulfonate |

*After gas-liquid diffusion for 10 days, a clear solution was obtained, and then volatilized at room temperature

### 3.3 Suspension Stirring at 5° C.

Approximately 20 mg of each K-13 methanesulfonate was weighed into an HPLC vial, 0.5 mL of a solvent was separately added, and the resulting suspensions were magnetically stirred at 5° C. for about 1 week, and solids were separated by centrifugation and tested by XRPD. A suspension stirring test was carried out at room temperature to obtain the crystalline form C of methanesulfonate. The experiments were as follows:

| Solvent (v:v) | Result |
|---|---|
| IPA | Crystalline form C of methanesulfonate |
| MEK | Crystalline form C of methanesulfonate |
| ACN | Crystalline form C of methanesulfonate |
| IPAc | Crystalline form C of methanesulfonate |
| MTBE | Crystalline form C of methanesulfonate |
| Acetone/EtOH (1:1) | Crystalline form C of methanesulfonate |
| DMF/Toluene (1:4) | Crystalline form C of methanesulfonate |

### 3.4 Suspension Stirring at Room Temperature

Approximately 20 mg of each K-13 methanesulfonate was weighed into an HPLC vial, 0.5 mL of a solvent was separately added, and the resulting suspensions were magnetically stirred at room temperature for about 1 week, and solids were separated by centrifugation and tested by XRPD to obtain the crystalline form C. The experiments were as follows:

| Solvent (v:v) | Result |
|---|---|
| MeOH | Crystalline form C of methanesulfonate |
| EtOH | Crystalline form C of methanesulfonate |
| Acetone | Crystalline form C of methanesulfonate |
| 2-MeTHF | Crystalline form C of methanesulfonate |
| EtOAc | Crystalline form C of methanesulfonate |
| MTBE | Crystalline form C of methanesulfonate |
| ACN | Crystalline form C of methanesulfonate |
| 1,4-Dioxane | Crystalline form C of methanesulfonate |
| THF | Crystalline form C of methanesulfonate |
| DCM | Crystalline form C of methanesulfonate |
| NMP/Anisole (1:4) | Crystalline form C of methanesulfonate |
| DMAc/n-Hexane (1:4) | Crystalline form C of methanesulfonate |
| DMSO/MEK (1:4) | Crystalline form C of methanesulfonate |
| Acetone/H₂O (986:14) | Crystalline form C of methanesulfonate |
| Acetone/H₂O (95:5) | Crystalline form C of methanesulfonate |
| Acetone/H₂O (86:14) | Crystalline form C of methanesulfonate |
| Acetone/H₂O (6:4) | Crystalline form C of methanesulfonate |

### 3.5 Suspension Stirring at 50° C.

Approximately 20 mg of each K-13 methanesulfonate was weighed into an HPLC vial, 0.5 mL of a solvent was separately added, and the resulting suspensions were magnetically stirred at 50° C. for about 3 days, and solids were separated by centrifugation and tested by XRPD. The crystalline form C was obtained. The experiments were as follows:

| Solvent (v:v) | Result |
|---|---|
| 2-MeTHF | Crystalline form C of methanesulfonate |
| 1-PrOH | Crystalline form C of methanesulfonate |
| MIBK | Crystalline form C of methanesulfonate |
| ACN | Crystalline form C of methanesulfonate |
| Anisole | Crystalline form C of methanesulfonate |
| n-BuOH | Crystalline form C of methanesulfonate |
| IPAC | Crystalline form C of methanesulfonate |
| CHCl₃/n-Heptane (1:4) | Crystalline form C of methanesulfonate |
| EtOH/H₂O (4:1) | Crystalline form C of methanesulfonate |
| NMP/EtOAc (1:4) | Crystalline form C of methanesulfonate |
| DMSO/Toluene (1:4) | Crystalline form C of methanesulfonate |

### 3.6 Temperature Cycle at 50-5° C.

Approximately 20 mg of each K-13 methanesulfonate was weighed into an HPLC vial, 0.5 mL of a solvent was separately added, and magnetic stirring was performed at 50° C. for 3 hours. Cooling was performed to 5° C. at a rate of 0.1° C./min, and stirring was performed at 5° C. for 0.5 hours; and the temperature was raised to 50° C. at a rate of 4.5° C./min, and stirring was performed at 50° C. for 0.5 hour. After the above steps was repeated twice, the temperature was reduced to 5° C. at a rate of 0.1° C./min and was kept at 5° C. A solid was collected and tested by XRPD to obtain the crystalline form C. The experiments were as follows:

| Solvent (v:v) | Result |
|---|---|
| Acetone | Crystalline form C of methanesulfonate |
| MEK | Crystalline form C of methanesulfonate |
| IPA | Crystalline form C of methanesulfonate |
| ACN | Crystalline form C of methanesulfonate |
| EtOH | Crystalline form C of methanesulfonate |
| Anisole | Crystalline form C of methanesulfonate |
| 2-MeTHF | Crystalline form C of methanesulfonate |
| EtOAc | Crystalline form C of methanesulfonate |
| DMF/MIBK (1:4) | Crystalline form C of methanesulfonate |
| CHCl₃/THF (1:1) | Crystalline form C of methanesulfonate |
| MeOH/CPME (1:1) | Crystalline form C of methanesulfonate |

### 3.7 Slow Cooling

Approximately 20 mg of each K-13 methanesulfonate was weighed into an HPLC vial, 1.0 mL of a solvent was separately added, filtering (using a 0.45 μm PTFE filter head) was performed after equilibration at 50° C. for about 2 hours with stirring, and a supernatant was taken. The resulting supernatant was placed in a biological incubator, cooled from 50° C. to 5° C. at 0.1° C./min and maintained at a constant temperature of 5° C. The precipitated solid was collected and tested by XRPD to obtain the crystalline form C. The experiments were as follows:

| Solvent (v:v) | Result |
|---|---|
| MeOH/THF (1:1) | Crystalline form C of methanesulfonate |

### 3.8 Anti-Solvent Addition

Approximately 20 mg of each K-13 methanesulfonate was weighed into a 20 mL vial, and after dissolution with 1.0-3.0 mL of a solvent, an anti-solvent was added dropwise to the clear solution while stirring until a solid was separated out, and addition was stopped if no solid was separated out after a total of 10.0 mL of the anti-solvent was added. Precipitated solids were separated by centrifugation and tested by XRPD. For systems with no solid precipitation after anti-solvent addition, volatilization was performed at room temperature to induce crystallization. The crystalline form C of methanesulfonate was obtained in an anti-solvent addition test. The experiments were as follows:

| | Anti- | |
|---|---|---|
| Solvent (mL) | solvent (mL) | Result |
| DCM (3.0) | MEK (7.0) | Crystalline form C of methanesulfonate |
| DCM (3.0) | Toluene (7.0) | Crystalline form C of methanesulfonate |
| MeOH (2.0) | MTBE (8.0) | Crystalline form C of methanesulfonate |
| MeOH (2.0) | Acetone (8.0) | Crystalline form C of methanesulfonate* |
| MeOH (2.0) | n-BuOH (8.0) | Crystalline form C of methanesulfonate* |
| MeOH (2.0) | Anisole (8.0) | Crystalline form C of methanesulfonate* |
| DMSO (1.0) | IPA (9.0) | Crystalline form C of methanesulfonate |
| NMP (1.0) | MEK (9.0) | Crystalline form C of methanesulfonate |

*the solution was still clear after anti-solvent addition and stirring for 3 days, and then volatilized at room temperature.

### 3.9 Salt Formation Reaction

About 4.8 mg of methanesulfonic acid was weighed into an HPLC vial, and diluted with 0.5 mL of a solvent, and then an equimolar amount of a K-13 free sample (about 20 mg) was weighed to be added into an HPLC vial, magnetic stirring was performed at room temperature for 5 days, and the resulting solid was collected and tested by XRPD. The crystalline form C of methanesulfonate was obtained in a salt formation reaction test. The experiments were as follows:

| Solvent | Result |
|---|---|
| EtOH | Crystalline form C of methanesulfonate |
| IPA | Crystalline form C of methanesulfonate |
| Acetone | Crystalline form C of methanesulfonate |
| EtOAc | Crystalline form C of methanesulfonate |
| MTBE | Crystalline form C of methanesulfonate |
| IPAc | Crystalline form C of methanesulfonate |
| THF | Crystalline form C of methanesulfonate |
| 2-MeTHF | Crystalline form C of methanesulfonate |
| ACN | Crystalline form C of methanesulfonate |

The results of XRPD showed that no change in crystalline form was found before and after air drying the crystalline form C of methanesulfonate at room temperature. The results of TGA showed that when the sample was heated from room temperature to 150° C., a weight loss of the sample was 2.5%. The results of DSC showed that overlapping endothermic peaks were observed at 234.6° C. and 241.9° C. (peak temperatures) for this sample.

### XRPD Peak-Searching Data for the Crystalline Form C of Methanesulfonate

| | | FWHM | | Rel. |
|---|---|---|---|---|
| Pos. [°2θ] | Height [cts] | Left [°2θ] | d-spacing [Å] | Int. [%] |
| 4.7539 | 613.91 | 0.1023 | 18.59 | 14.85 |
| 9.4706 | 313.75 | 0.0768 | 9.34 | 7.59 |
| 10.4270 | 913.69 | 0.0768 | 8.48 | 22.10 |
| 10.7019 | 3018.45 | 0.1023 | 8.27 | 72.99 |
| 11.0858 | 707.21 | 0.1023 | 7.98 | 17.10 |
| 12.6174 | 276.86 | 0.1023 | 7.02 | 6.70 |
| 12.9503 | 515.32 | 0.1023 | 6.84 | 12.46 |
| 14.0934 | 623.94 | 0.1023 | 6.28 | 15.09 |
| 15.7866 | 191.79 | 0.1023 | 5.61 | 4.64 |
| 16.5463 | 4135.16 | 0.1535 | 5.36 | 100.00 |
| 17.0992 | 319.61 | 0.0768 | 5.19 | 7.73 |
| 17.6406 | 621.28 | 0.0768 | 5.03 | 15.02 |
| 17.8994 | 1835.25 | 0.1535 | 4.96 | 44.38 |
| 18.9721 | 1268.18 | 0.1023 | 4.68 | 30.67 |
| 19.6476 | 942.01 | 0.1023 | 4.52 | 22.78 |
| 19.8422 | 288.56 | 0.0768 | 4.47 | 6.98 |
| 20.2635 | 95.77 | 0.1535 | 4.38 | 2.32 |
| 21.4424 | 648.85 | 0.1023 | 4.14 | 15.69 |
| 21.7511 | 2429.52 | 0.1791 | 4.09 | 58.75 |
| 22.1721 | 1890.91 | 0.1535 | 4.01 | 45.73 |
| 22.8579 | 1163.98 | 0.1535 | 3.89 | 28.15 |
| 23.3591 | 1050.17 | 0.1279 | 3.81 | 25.40 |
| 23.8135 | 423.75 | 0.1279 | 3.74 | 10.25 |
| 24.2008 | 430.58 | 0.1023 | 3.68 | 10.41 |
| 25.2336 | 522.15 | 0.0768 | 3.53 | 12.63 |
| 25.5054 | 2581.42 | 0.1279 | 3.49 | 62.43 |
| 25.8347 | 1237.93 | 0.1023 | 3.45 | 29.94 |
| 26.3811 | 3198.82 | 0.1535 | 3.38 | 77.36 |
| 27.1392 | 636.57 | 0.1023 | 3.29 | 15.39 |
| 27.6134 | 826.20 | 0.1279 | 3.23 | 19.98 |
| 29.1780 | 355.86 | 0.1023 | 3.06 | 8.61 |
| 30.4842 | 96.40 | 0.1535 | 2.93 | 2.33 |
| 31.0025 | 93.13 | 0.1535 | 2.88 | 2.25 |
| 32.0069 | 125.66 | 0.1535 | 2.80 | 3.04 |
| 32.8408 | 119.23 | 0.1535 | 2.73 | 2.88 |
| 35.3265 | 82.41 | 0.1535 | 2.54 | 1.99 |
| 36.1865 | 99.70 | 0.1535 | 2.48 | 2.41 |
| 38.4105 | 144.67 | 0.1279 | 2.34 | 3.50 |

### 3.10 Investigation on the Properties of the Crystalline Form C

### Hygroscopicity

The hygroscopicity of the anhydrate crystalline form C of methanesulfonate was evaluated by a DVS test at 0% RH-95% RH at 25° C. DVS and XRPD characterization results showed that the hygroscopic weight gain of a sample, i.e., the crystalline form C of methanesulfonate was about 0.46% at 25° C./80% RH, indicating its slight hygroscopicity (a classification standard for hygroscopicity refers to the 2015 edition of the “Chinese Pharmacopoeia”), and the results of XRPD showed that the crystalline form of the sample remained unchanged after the DVS test. The experiment showed that the hygroscopicity of the crystalline form C was better than that of the crystalline form A and the crystalline form B.

### Solid State Stability of the Crystalline Form C of Methanesulfonate

There was no significant decrease in HPLC purity after the sample, i.e., the crystalline form C of methanesulfonate was placed under a closed condition at 60° C. for 24 hours and an open condition at 25° C./60% RH for 1 week or 4 weeks. The purity decreased from 99.22 area % to 98.80 area % after the crystalline form C was placed under an open condition in an environment of 40° C./75% RH for 4 weeks, with a 0.38% increase in the impurity at RRT of 0.81. HPLC data for samples before and after the stability test are summarized below. Comparison of XRPD results of samples before and after the stability test showed that there was no change in crystalline form of the samples after the stability test. The experiment showed that the solid state stability of the crystalline form C was better than that of the crystalline form A and the crystalline form B.

| | | Purity, area % | | | | | | |
|---|---|---|---|---|---|---|---|---|
| | | | 60° C./closed | | | | | |
| | | | condition/24 | | 25° C./60% RH | | 40° C./75% RH | |
| #Peak | RRT | Start | hours | Start* | 1 week* | 4 weeks | 1 week* | 4 weeks |
| 1 | 0.42 | 0.03 | 0.03 | 0.02 | 0.02 | 0.02 | 0.03 | 0.03 |
| 2 | 0.60-0.61 | 0.05 | 0.05 | 0.03 | 0.04 | 0.05 | 0.04 | 0.08 |
| 3 | 0.81 | 0.54 | 0.55 | 0.48 | 0.55 | 0.54 | 0.69 | 0.86 |
| 4 | 0.93 | 0.05 | 0.05 | 0.05 | 0.05 | 0.05 | 0.04 | 0.05 |
| 5 | 1.00 | 99.13 | 99.12 | 99.22 | 99.15 | 99.14 | 99.02 | 98.80 |
| 6 | 1.10 | 0.15 | 0.15 | 0.14 | 0.14 | 0.14 | 0.13 | 0.13 |
| 7 | 1.27 | 0.06 | 0.05 | 0.06 | 0.06 | 0.06 | 0.06 | 0.05 |

*Samples were tested simultaneously with samples with stability after 4 weeks after storage at −20° C.

The crystalline form C of methanesulfonate had slight hygroscopicity and there was no change in crystalline form after the DVS test. No significant change in purity was observed after the crystalline form C was placed under a closed condition at 60° C. for 24 hours and an open condition at 25° C./60% RH for 4 weeks, the purity was decreased by 0.42 area % after the crystalline form C was placed under an open condition in an environment of 40° C./75% RH for 4 weeks, and there was no change in crystalline form of the sample after the stability test.

### Example 3 Test of Influencing Factors of Crystalline Forms A, B and C

| | | Total impurity % | | | |
|---|---|---|---|---|---|
| | Placement conditions | 0 day | 5 days | 10 days | 30 days |
| Crystalline | High temperature | 0.91% | 4.50% | 6.96% | / |
| form A | (60° C.) | | | | |
| | High humidity | 0.91% | 0.92% | 0.99% | 1.25% |
| | (RH75%) | | | | |
| | Illumination | 0.91% | 1.89% | 3.70% | 4.21% |
| | (5000 + 500 lux) | | | | |
| Crystalline | High temperature | 0.86% | 1.00% | 0.98% | / |
| form B | (60° C.) | | | | |
| | High humidity | 0.86% | 1.03% | 1.09% | 1.12% |
| | (RH75%) | | | | |
| | Illumination | 0.86% | 0.94% | 1.12% | 1.33% |
| | (5000 + 500 lux) | | | | |
| Crystalline | High temperature | 0.66% | / | 0.81% | 0.85% |
| form C | (60° C.) | | | | |
| | High humidity | 0.66% | / | 0.75% | 0.73% |
| | (RH92.5%) | | | | |
| | Illumination | 0.66% | / | 0.83% | 0.89% |
| | (5000 + 500 lux) | | | | |

Taken together, the anhydrous crystalline form C of methanesulfonate is a dominant crystalline form.

### Example 4

Comparison of oral absorption of K-13 (free base) and its methanesulfonate (N-(3-fluorophenyl)-6-(6. 7-dimethoxyquinolin-4-oxy)-3.4-dihydroquinoline-1(2H)-carboxamide methanesulfonate)

Individual and mean plasma concentration-time data for K-13-1 after oral
administration of 10 mg · kg⁻¹ of K-13-1 (free base) to BALB/c mice

| | | Sampling | Concentration | | |
|---|---|---|---|---|---|
| Dose | Dose | Time | (ng · mL⁻¹) | | Mean |
| (mg · kg⁻¹) | Route | (h) | PO-1 | PO-2 | (ng · mL⁻¹) |
| 10 | PO | 0 | 0 | 0 | 0 |
| | | 0.083 | 29.6 | 46.6 | 38.1 |
| | | 0.25 | 186 | 215 | 201 |
| | | 0.5 | 480 | 865 | 673 |
| | | 1 | 551 | 420 | 485 |
| | | 2 | 136 | 485 | 311 |
| | | 4 | 220 | 395 | 308 |
| | | 8 | 97.2 | 58.8 | 78.0 |
| | | 24 | BLOQ | 4.65 | 4.65 |

| PK Parameters | Unit | |
|---|---|---|
| Cₘₐₓ | ng · mL⁻¹ | 673 |
| Tₘₐₓ | h | 0.50 |
| Kₑₗ | h⁻¹ | 0.200 |
| T_{1/2} | h | 3.5 |
| AUC₀₋ₜ | ng · h · mL⁻¹ | 2870 |
| AUC_{0-inf} | ng · h · mL⁻¹ | 2894 |
| F | % | 20 |

Individual and mean plasma concentration-time data for
K-13-1 after administration of 10 mg · kg⁻¹ of K-13-MS-1
(a methanesulfonate Formulation) to BALB/c mice

| | | Sampling | Concentration | | |
|---|---|---|---|---|---|
| Dose | Dose | Time | (ng · mL⁻¹) | | Mean |
| (mg · kg⁻¹) | Route | (h) | PO-1 | PO-2 | (ng · mL⁻¹) |
| 10 | PO | 0 | 0 | 0 | 0 |
| | | 0.083 | 431 | 414 | 423 |
| | | 0.25 | 2175 | 2059 | 2117 |
| | | 0.5 | 1868 | 1775 | 1822 |
| | | 1 | 1824 | 1317 | 1571 |
| | | 2 | 922 | 1319 | 1121 |
| | | 4 | 884 | 981 | 932 |
| | | 8 | 130 | 89.0 | 109 |
| | | 24 | BLOQ | BLOQ | BLOQ |

| PK Parameters | Unit | |
|---|---|---|
| Cₘₐₓ | ng · mL⁻¹ | 2117 |
| Tₘₐₓ | h | 0.25 |
| Kₑₗ | h⁻¹ | 0.364 |
| T_{1/2} | h | 1.9 |
| AUC₀₋ₜ | ng · h · mL⁻¹ | 7051 |
| AUC_{0-inf} | ng · h · mL⁻¹ | 7350 |
| *F | % | 50 |

As can be seen from the above tables, when a free base was prepared into methanesulfonate, the plasma concentration was significantly increased, the in vivo exposure Cₘₐₓ and AUCₗₐₛₜ were increased by 3.14 times and 2.46 times, respectively, and the oral bioavailability was significantly increased (from 20% to 50%), satisfying the needs of the human body and contributing to the efficacy of the drug.

The above are only better examples of the present disclosure, and are not intended to limit the present disclosure, and any modification, equivalent replacement, improvement and the like made within the spirit and principle of the present disclosure should be included in the protection scope of the present disclosure.

1. A compound N-(3-fluorophenyl)-6-(6,7-dimethoxyquinolin-4-oxy)-3,4-dihydroquinoline-1(2H)-carboxamide methanesulfonate, having the following structural formula:

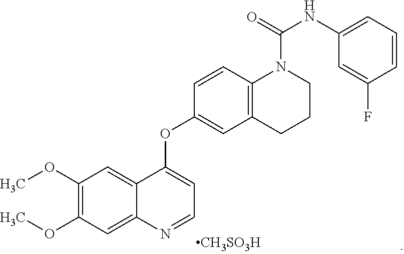

2. A crystalline form A of a compound N-(3-fluorophenyl)-6-(6,7-dimethoxyquinolin-4-oxy)-3,4-dihydroquinoline-1(2H)-carboxamide methanesulfonate, wherein an X-ray powder diffraction pattern using Cu-Kα radiation shows characteristic peaks at 2θ of 6.5°±0.2°, 10.0°±0.2°, 15.2°±0.2°, 17.2°±0.2°, 19.8°±0.2°, and 24.3°±0.2°.

3. A method for preparing the crystalline form A according to claim 2, comprising:
(a) weighting the compound N-(3-fluorophenyl)-6-(6,7-dimethoxyquinolin-4-oxy)-3,4-dihydroquinoline-1(2H)-carboxamide methanesulfonate into a vial, dissolving the compound by adding a solvent to be clear, and slowly volatilizing the solvent at room temperature.

4. The method according to claim 3, wherein in the step (a), the solvent is Acetone/H₂O, EtOH/H₂O, methanol/water, THF/H₂O or ACN/H₂O in a volume ratio of 3-4:1, and a volume (ml) of the solvent used is 0.1-0.5 times a weight (mg) of the compound.

5. A crystalline form B of a compound N-(3-fluorophenyl)-6-(6,7-dimethoxyquinolin-4-oxy)-3,4-dihydroquinoline-1(2H)-carboxamide methanesulfonate, wherein an X-ray powder diffraction pattern using Cu-Kα radiation shows characteristic peaks at 2θ of 5.3°±0.2°, 9.6°±0.2°, 14.5°±0.2°, 21.5°±0.2°, 24.1°±0.2°, and 27.0°±0.2°.

6. A method for preparing the crystalline form B according to claim 5, comprising:
(a) weighting the compound N-(3-fluorophenyl)-6-(6,7-dimethoxyquinolin-4-oxy)-3,4-dihydroquinoline-1(2H)-carboxamide methanesulfonate into a vial, dissolving the compound by adding a solvent, performing filtering, and allowing a filtrate and an anti-solvent to each independently coexist in a closed space and stand at room temperature.

7. The method according to claim 6, wherein in the step (a), the solvent is selected from DMF, CHCl₃, and methanol, the anti-solvent is selected from Acetone, THF, and MEK, a volume (ml) of the solvent used is 0.04-0.1 times a weight (mg) of the compound, and a volume (ml) of the anti-solvent used is 1-5 times the volume (ml) of the solvent.

8. A crystalline form C of a compound N-(3-fluorophenyl)-6-(6,7-dimethoxyquinolin-4-oxy)-3,4-dihydroquinoline-1(2H)-carboxamide methanesulfonate, wherein an X-ray powder diffraction pattern using Cu-Kα radiation shows characteristic peaks at 2θ of 4.7°±0.2°, 10.4°±0.2°, 15.7°±0.2°, 19.8°±0.2°, 22.8°±0.2°, and 25.8°±0.2°.

9. A method for preparing the crystalline form C according to claim 8, comprising:
(a) weighting the compound N-(3-fluorophenyl)-6-(6,7-dimethoxyquinolin-4-oxy)-3,4-dihydroquinoline-1(2H)-carboxamide methanesulfonate into a vial, adding a solvent, and performing pulping.

10. The method according to claim 9, wherein in the step (a), the solvent is selected from IPA, MEK, IPAc, 1-PrOH, ethanol, methanol, Acetone, 2-MeTHF, EtOAc, MTBE, ACN, 1,4-Dioxane, THF, DCM, MIBK, Anisole, n-BuOH, or a mixed solvent NMP/Anisole, DMAc/n-Hexane, DMSO/MEK, Acetone/H₂O, Acetone/EtOH, DMF/Toluene, CHCl₃/n-Heptane, EtOH/H₂O, NMP/EtOAc, DMSO/Toluene, DMF/MIBK, CHCl₃/THF, or MeOH/CPME, a volume (ml) of the solvent or the mixed solvent used is 0.025 times a weight (g) of the compound, a volume ratio (ml/ml) of Acetone to H₂O in the mixed solvent is 1.5:1-75:1, and a volume ratio in other mixed solvents is 1:4-4:1.

11. A method for preparing the crystalline form C according to claim 8, comprising:
(a) weighting the compound N-(3-fluorophenyl)-6-(6,7-dimethoxyquinolin-4-oxy)-3,4-dihydroquinoline-1(2H)-carboxamide methanesulfonate into a vial, dissolving the compound by adding a solvent, performing filtering, and allowing a filtrate and an anti-solvent to each independently coexist in a closed space and stand at room temperature.

12. The method according to claim 11, wherein in the step (a), the solvent is selected from DMF, CHCl₃, and methanol, the anti-solvent is selected from Ethyl formate, IPAc, IPA, MTBE, and ACN, a volume (ml) of the solvent used is 0.04-0.1 times a weight (mg) of the compound, and a volume (ml) of the anti-solvent used is 1-5 times the volume (ml) of the solvent.

13. A method for preparing the crystalline form C according to claim 8, comprising:
(a) weighting the compound N-(3-fluorophenyl)-6-(6,7-dimethoxyquinolin-4-oxy)-3,4-dihydroquinoline-1(2H)-carboxamide methanesulfonate into a vial, dissolving the compound by adding a solvent, adding an anti-solvent, and performing stirring until a solid is separated out.

14. The method according to claim 13, wherein in the step (a), the solvent is selected from DMSO, NMP, methanol, and DCM, the anti-solvent is selected from Toluene, IPA, MEK, MTBE, Acetone, n-BuOH, and Anisole, a volume (ml) of the solvent used is 0.05-0.15 times a weight (mg) of the compound, and a volume (ml) of the anti-solvent used is 2-10 times the volume (ml) of the solvent.
